(19) United States
(12) Patent Application Publication
(10) Pub. No.: US 2026/0306813 A1
(43) Pub. Date: Oct. 8, 2026

(54) MICRONEEDLE MANUFACTURING METHOD, MICRONEEDLE, AND MICRONEEDLE UNIT

(71) Applicants: ASTI CORPORATION, Hamamatsu-shi, Shizuoka (JP); KINKI SEIKO CO., LTD., Nagahama-shi, Shiga (JP)

(72) Inventors: Noriyuki OGAI, Hamamatsu-shi, Shizuoka (JP); Yasuhiro TODA, Hamamatsu-shi, Shizuoka (JP); Isamu NONAKA, Hamamatsu-shi, Shizuoka (JP); Kimiaki IWAHORI, Hamamatsu-shi, Shizuoka (JP); Yasuhiro HATAZAWA, Nagahama-shi, Shiga (JP); Kosuke HATAZAWA, Nagahama-shi, Shiga (JP); Masahiro TERAMOTO, Nagahama-shi, Shiga (JP)

(21) Appl. No.: 19/482,778

(22) PCT Filed: May 10, 2024
(86) PCT No.: PCT/JP2024/017445

§ 371 Date: Nov. 10, 2025

(30) Foreign Application Priority Data
May 12, 2023 (JP) 2023-079322

(51) Int. Cl. A61M 37/00

(52) U.S. Cl. CPC A61M 37/0015; A61M 2037/0023; A61M 2037/003; A61M 2037/0053

(57) ABSTRACT

A microneedle manufacturing method for manufacturing a microneedle provided with a vertical hole as a liquid drug flow channel and a horizontal hole as a liquid drug discharge port connected to the vertical hole, wherein a slide pin for molding the horizontal hole is formed in a molding die; a core pin for molding the vertical hole is brought into contact with the formed slide pin; a resin is filled in the molding die in the state described above; and after a curing period, the core pin is retracted and the slide pin is pulled out so as to integrally mold the microneedle provided with the vertical hole as the liquid drug flow channel and the horizontal hole as the liquid drug discharge port connected to the vertical hole in a penetrating state.

### RELATED APPLICATIONS

The present application is National Phase of International Application No. PCT/JP2024/017445 filed May 10, 2024, and claims priority from Japanese Application No. 2023-079322, filed May 12, 2023.

### TECHNICAL FIELD

The present invention relates to a microneedle manufacturing method, a microneedle, and a microneedle unit, and in particular, the present invention relates to those allowing easy manufacturing of a microneedle provided with a horizontal hole in a penetrated state serving as a medical solution discharge port.

### BACKGROUND ART

Generally, in a case of a hollow microneedle made of resin, a vertical hole has been provided at a center of a tip, for performing subcutaneous or intradermal injection of medical solution. In a case of such a conventional hollow microneedle made of resin, there have been problems that the mechanical strength is poor and the puncture is difficult.

Thus, a horizontal-hole type of hollow microneedle has been proposed. As an example of disclosure of configuration of this type of hollow microneedle, there are Patent Document 1 and Patent Document 2.

First, according to an invention described in Patent Document 1, a microneedle unit has a configuration that a first divided element and a second divided element are bonded to each other, and a medical solution discharge port is provided in a horizontal direction. Accordingly, the mechanical strength of the tip is secured, and furthermore, during puncturing, the clogging by skin, etc., is prevented, and therefore, it is possible to perform intradermal or subcutaneous injection of medical solution effectively.

Note that, the patent owner of Patent Document 1 is the applicant of the present invention.

Next, in a case of a hollow microneedle array according to an invention described in Patent Document 2, an orifice is provided substantially in a horizontal direction on a slanted surface of a molded product.

### REFERENCE DOCUMENTS OF CONVENTIONAL ART

### Patent Document(s)

Patent Document 1: Official Gazette, Japan Patent No. 6347960.

Patent Document 2: Official Gazette, Japan Unexamined Patent Publication (Translation of PCT Application) No. 2012-523270A.

### SUMMARY OF THE INVENTION

### Problems to be Solved by Invention

However, the configurations according to the conventional arts described above have following problems.

First, in the case of invention described in Patent Document 1, the first divided element and the second divided element must be bonded to each other, and consequently, there have been problems of difficulty in mass production precisely and by securing airtightness, and furthermore, the number of manufacturing processes becomes larger, and the production cost becomes expensive.

Moreover, there has been a problem that a shape of a medical solution discharge port is limited.

Furthermore, a needle base is required for attaching a microneedle unit to a syringe, and such a needle base must be formed as a separate component, and there has been a problem that the production cost becomes more expensive.

Moreover, in the case of invention described in Patent Document 2, essentially, the orientation of orifice is not definitely horizontal, and there has been a problem that the effect of horizontal orientation cannot be achieved sufficiently.

Moreover, during manufacturing, a plurality of plates is required, and therefore, likewise the problems of invention described in Patent Document 1, there have been problems that complicated working and adjustments are required.

Moreover, because an opening is formed by contact of a penetration pin with a cavity, burrs are generated by breakage and wear, and in addition, there have been further problems that the shapes of needle and hole are limited, and that some areas become structurally weak. Consequently, it is very difficult to form the openings of all needles uniformly.

In the light of the above problem, it is an object of the present invention to provide a microneedle manufacturing method, and a microneedle and a microneedle unit, allowing easy manufacturing of a microneedle provided with a horizontal hole in a penetrated state serving as a medical solution discharge port.

### Means to Solve the Problem

To achieve the objects mentioned above, according to Claim 1 of the present invention, a microneedle manufacturing method, for manufacturing a microneedle provided with a vertical hole serving as a medical solution flow path, and a connected horizontal hole serving as a medical solution discharge port in a penetrated state, is characterized in that: a slide pin, for forming the horizontal hole in a mold, is caused to appear; a core pin, for forming the vertical hole, is caused to abut on the appeared slide pin; resin is filled in the mold in the appeared and abutting states; and after passing of a curing period, the core pin is retracted, and also the slide pin is withdrawn. Consequently, the microneedle provided with the vertical hole serving as the medical solution flow path, and the connected horizontal hole serving as the medical solution discharge port in the penetrated state, is molded integrally.

Moreover, according to the microneedle manufacturing method of Claim 2, with regard to the microneedle manufacturing method as claimed in Claim 1: the mold is configured by a pair of cores arranged to be joinable and separatable; the slide pin is caused to appear in a clearance between the pair of cores in a state separate from each other; the slide pin is sandwiched by moving the pair of cores close to each other; the core pin is caused to appear and abut on the slide pin; the resin is filled into the clearance between the pair of cores in the appeared and abutting states; and after passing of the curing period, the core pin is retracted, and also the slide pin is withdrawn. Consequently, the microneedle provided with the vertical hole serving as the medical solution flow path, and the connected horizontal hole serving as the medical solution discharge port in the penetrated state, is molded integrally.

Moreover, according to the microneedle manufacturing method of Claim 3, with regard to the microneedle manufacturing method as claimed in Claim 2, the pair of cores is a pair of slide cores arranged to be joinable and separatable in a direction orthogonal to an axis direction of the microneedle, and the microneedle is molded by this pair of slide cores.

Moreover, according to the microneedle manufacturing method of Claim 4, with regard to the microneedle manufacturing method as claimed in Claim 3: a core insert is arranged between the pair of slide cores; and the core pin appears and disappears by penetrating through the core insert.

Moreover, according to the microneedle manufacturing method of Claim 5, with regard to the microneedle manufacturing method as claimed in Claim 4, in each of the pair of slide cores, a recess is formed for accommodating the slide pin, and a cavity is formed for accommodating the core insert in a state that the core pin is caused to appear.

Moreover, according to the microneedle manufacturing method of Claim 6, with regard to the microneedle manufacturing method as claimed in Claim 5: the pair of slide cores is set to be in a size for molding an area extending from the microneedle to a needle base; the core insert is also set to be in a size for molding the area extending from the microneedle to the needle base; and the area extending from the microneedle to the needle base is molded integrally as a microneedle unit by using the pair of slide cores and the core insert.

Moreover, according to the microneedle manufacturing method of Claim 7, with regard to the microneedle manufacturing method as claimed in Claim 4: a plurality of core pins is arranged in a single row in the core insert; a cavity is provided in the slide core, for accommodating the core insert in a state that the plurality of core pins is caused to appear; and a plurality of microneedles is molded integrally in a state that the plurality of microneedles is arranged in the single row by using the pair of slide cores and the core insert.

Moreover, according to the microneedle manufacturing method of Claim 8, with regard to the microneedle manufacturing method as claimed in Claim 7, the horizontal holes of the plurality of microneedles are formed by single straight penetration of the single slide pin.

Moreover, according to the microneedle manufacturing method of Claim 9, with regard to the microneedle manufacturing method as claimed in Claim 2, the pair of cores is a cavity insert upper plate and a cavity insert lower plate arranged to be joinable and separatable in an axis direction of the microneedle, and the microneedle is molded by the cavity insert upper plate and the cavity insert lower plate.

Moreover, according to the microneedle manufacturing method of Claim 10, with regard to the microneedle manufacturing method as claimed in Claim 9, a core insert is arranged to be movable in the axis direction of the microneedle below the cavity insert lower plate.

Moreover, a microneedle according to Claim 11 is provided with a vertical hole serving as a medical solution flow path, and a connected horizontal hole serving as a medical solution discharge port in a penetrated state, and is molded integrally.

Moreover, according to the microneedle of Claim 12, with regard to the microneedle as claimed in Claim 11, a cross section of the horizontal hole is in a circular shape.

Moreover, according to the microneedle of Claim 13, with regard to the microneedle as claimed in Claim 11: the microneedle includes a foundation, a neck section provided on a tip side of the foundation, and a boring section provided on a tip side of the neck section; and the horizontal hole is provided in a lower part of the boring section.

Moreover, according to the microneedle of Claim 14, with regard to the microneedle as claimed in Claim 13: the boring section is a pyramid section; and the horizontal hole is provided by penetrating through a line connecting a pair of edges of the pyramid section.

Moreover, according to the microneedle of Claim 15, with regard to the microneedle as claimed in Claim 14, an end part of each of the edges of the pyramid section on a side of the neck section is formed in an arc shape protruding outwardly.

Moreover, according to the microneedle of Claim 16, with regard to the microneedle as claimed in Claim 13, a lower part of the foundation is formed to be in a larger diameter than that of the neck section, and the diameter decreases gradually toward the neck section.

Moreover, according to the microneedle of Claim 17, with regard to the microneedle as claimed in Claim 13, the vertical hole is formed extending from the foundation to the neck section, and becomes narrower gradually from the foundation to the neck section.

And moreover, a microneedle unit according to Claim 18 is provided with: the microneedle as claimed in any one claim among claim 11 to claim 17; and a needle base, integrally molded on a base end side of the microneedle, and a tip of a cylindrical body of a syringe is coupled with the base end side.

### Effect of the Invention

As described above, according to Claim 1 of the present invention, a microneedle manufacturing method, for manufacturing a microneedle provided with a vertical hole serving as a medical solution flow path, and a connected horizontal hole serving as a medical solution discharge port in a penetrated state, is characterized in that: a slide pin, for forming the horizontal hole in a mold, is caused to appear; a core pin, for forming the vertical hole, is caused to abut on the appeared slide pin; resin is filled in the mold in the appeared and abutting states; and after passing of a curing period, the core pin is retracted, and also the slide pin is withdrawn. Consequently, the microneedle provided with the vertical hole serving as the medical solution flow path, and the connected horizontal hole serving as the medical solution discharge port in the penetrated state, is molded integrally. Therefore, it is possible to manufacture the microneedle easily, provided with the vertical hole serving as the medical solution flow path, and the connected horizontal hole serving as the medical solution discharge port in the penetrated state.

Also, during manufacturing, because the both ends of the slide pin are held strongly by the molds, the rigidity of the slide pin is secured, the improper positioning and deformation are prevented, the horizontal hole can formed precisely, and there is no generation of burrs at a boundary between the slide pin and the core pin.

Moreover, with regard to the slide pin, because the core pin abuts thereon, this configuration also serves for the effects that, the rigidity is secured, the improper positioning and deformation are prevented, the horizontal hole can formed precisely, and there is no generation of burrs at a boundary from the slide pin.

Moreover, the above affects can also be achieved with regard to the core pin, and because of being abutted by the slide pin, the improper positioning and deformation due to resin pressure are prevented, the horizontal hole can formed precisely, and there is no generation of burrs at a boundary from the slide pin.

Moreover, according to the microneedle manufacturing method of Claim 2, with regard to the microneedle manufacturing method as claimed in Claim 1: the mold is configured by a pair of cores arranged to be joinable and separatable; the slide pin is caused to appear in a clearance between the pair of cores in a state separate from each other; the slide pin is sandwiched by moving the pair of cores close to each other; the core pin is caused to appear and abut on the slide pin; the resin is filled into the clearance between the pair of cores in the appeared and abutting states; and after passing of the curing period, the core pin is retracted, and also the slide pin is withdrawn. Consequently, the microneedle provided with the vertical hole serving as the medical solution flow path, and the connected horizontal hole serving as the medical solution discharge port in the penetrated state, is molded integrally. Therefore, the above effects can be achieved securely.

Moreover, because the mold is configured by the pair of cores, the configuration is simple.

Moreover, according to the microneedle manufacturing method of Claim 3, with regard to the microneedle manufacturing method as claimed in Claim 2, the pair of cores is a pair of slide cores arranged to be joinable and separatable in a direction orthogonal to an axis direction of the microneedle, and the microneedle is molded by this pair of slide cores. Therefore, it is possible to manufacture the microneedles in a single row precisely.

Moreover, according to the microneedle manufacturing method of Claim 4, with regard to the microneedle manufacturing method as claimed in Claim 3: a core insert is arranged between the pair of slide cores; and the core pin appears and disappears by penetrating through the core insert. Therefore, the above effects can be achieved securely.

Moreover, the appearing and disappearing of the core pin can be performed easily, and the abutting state can be adjusted easily.

Moreover, according to the microneedle manufacturing method of Claim 5, with regard to the microneedle manufacturing method as claimed in Claim 4, in each of the pair of slide cores, a recess is formed for accommodating the slide pin, and a cavity is formed for accommodating the core insert in a state that the core pin is caused to appear. Therefore, the above effects can be achieved securely.

Also, during manufacturing, because the both ends of the slide pin are held strongly by the recesses of the slide cores, the rigidity of the slide pin is secured, the improper positioning and deformation are prevented, the horizontal hole can formed precisely, and there is no generation of burrs at a boundary between the slide pin and the core pin.

Moreover, it is possible to hold the slide pin securely by the recesses of the pair of slide cores.

Moreover, according to the microneedle manufacturing method of Claim 6, with regard to the microneedle manufacturing method as claimed in Claim 5: the pair of slide cores is set to be in a size for molding an area extending from the microneedle to a needle base; the core insert is also set to be in a size for molding the area extending from the microneedle to the needle base; and the area extending from the microneedle to the needle base is molded integrally as a microneedle unit by using the pair of slide cores and the core insert. Therefore, the above effects can be achieved securely.

Moreover, it is possible to mold the area extending from the microneedle to the needle base as the microneedle unit easily.

Moreover, according to the microneedle manufacturing method of Claim 7, with regard to the microneedle manufacturing method as claimed inClaim 4: a plurality of core pins is arranged in a single row in the core insert; a cavity is provided in the slide core, for accommodating the core insert in a state that the plurality of core pins is caused to appear; and a plurality of microneedles is molded integrally in a state that the plurality of microneedles is arranged in the single row by using the pair of slide cores and the core insert. Therefore, the above effects can be achieved securely.

Moreover, it is possible to mold the plurality of microneedles arranged in the single row easily.

Moreover, according to the microneedle manufacturing method of Claim 8, with regard to the microneedle manufacturing method as claimed in Claim 7, the horizontal holes of the plurality of microneedles are formed by single straight penetration of the single slide pin. Therefore, it is possible to form the horizontal hole in each of the plurality of microneedles easily.

Moreover, according to the microneedle manufacturing method of Claim 9, with regard to the microneedle manufacturing method as claimed in Claim 2, the pair of cores is a cavity insert upper plate and a cavity insert lower plate arranged to be joinable and separatable in an axis direction of the microneedle, and the microneedle is molded by the cavity insert upper plate and the cavity insert lower plate. Therefore, it is possible to manufacture the microneedles in multiple rows precisely.

Moreover, according to the microneedle manufacturing method of Claim 10, with regard to the microneedle manufacturing method as claimed in Claim 9, a core insert is arranged to be movable in the axis direction of the microneedle below the cavity insert lower plate. Therefore, it is possible to manufacture the microneedle without extending a space in the direction orthogonal to the axis direction of the microneedle.

Moreover, a microneedle according to Claim 11 is provided with a vertical hole serving as a medical solution flow path, and a connected horizontal hole serving as a medical solution discharge port in a penetrated state, and is molded integrally. Therefore, the mechanical strength is excellent, and it is possible to perform puncture and injection of medical solution securely.

Moreover, because of large opening area, the injection resistance can be reduced, and it is possible to perform efficient, shallow and broad diffusion of medical agent in the skin.

Moreover, according to the microneedle of Claim 12, with regard to the microneedle as claimed in Claim 11, a cross section of the horizontal hole is in a circular shape. Therefore, it is possible to inject the medical solution efficiently.

Moreover, according to the microneedle of Claim 13, with regard to the microneedle as claimed in Claim 11: the microneedle includes a foundation, a neck section provided on a tip side of the foundation, and a boring section provided on a tip side of the neck section; and the horizontal hole is provided in a lower part of the boring section. Therefore, the puncture can be performed securely, and the injection of medical solution can be performed securely.

Moreover, it is possible to improve the puncturing performance by the boring section, and moreover, it is possible to secure the needle strength and improve the molding efficiency by the foundation, and furthermore, because of extension effect of skin, the puncture can be performed more easily. Moreover, because the puncture stops at the foundation, the depth of puncture is maintained constantly.

Moreover, according to the microneedle of Claim 14, with regard to the microneedle as claimed in Claim 13: the boring section is a pyramid section; and the horizontal hole is provided by penetrating through a line connecting a pair of edges of the pyramid section. Therefore, the puncture can be performed securely, and furthermore, the injection of medical solution can be performed securely. Moreover, it is possible to improve the puncturing performance by the edges (ridgelines).

Moreover, according to the microneedle of Claim 15, with regard to the microneedle as claimed in Claim 14, an end part of each of the edges of the pyramid section on a side of the neck section is formed in an arc shape protruding outwardly. Therefore, during withdrawal after puncture, damage to the skin can be prevented.

Moreover, according to the microneedle of Claim 16, with regard to the microneedle as claimed in Claim 13, a lower part of the foundation is formed to be in a larger diameter than that of the neck section, and the diameter decreases gradually toward the neck section. Therefore, the puncture can be performed easily, and the depth of puncture can be maintained constantly.

Moreover, according to the microneedle of Claim 17, with regard to the microneedle as claimed in Claim 13, the vertical hole is formed extending from the foundation to the neck section, and becomes narrower gradually from the foundation to the neck section. Therefore, it is possible to inject the medical solution efficiently.

And moreover, a microneedle unit according to Claim 18 is provided with: the microneedle as claimed in any one claim among claim 11 to claim 17; and a needle base, integrally molded on a base end side of the microneedle, and a tip of a cylindrical body of a syringe is coupled with the base end side. Therefore, a portion extending from the microneedle to the needle base becomes a single component, and it is possible to simplify the configuration, and facilitate components management.

### BRIEF DESCRIPTION OF DRAWINGS

FIG. 1 is a perspective view showing a configuration of a microneedle unit according to a first embodiment of the present invention.

FIG. 2 is an expanded perspective view showing a section II of FIG. 1 according to the first embodiment of the present invention.

FIG. 3 is a front view of the microneedle unit according to the first embodiment of the present invention.

FIG. 4 is an expanded perspective view showing a section IV of FIG. 3 according to the first embodiment of the present invention.

FIG. 5 is a side view of the microneedle unit according to the first embodiment of the present invention.

FIG. 6 is an expanded side view showing a section VI of FIG. 5 according to the first embodiment of the present invention.

FIG. 7 is a front view of a microneedle according to the first embodiment of the present invention.

FIG. 8 is a perspective view of the microneedle according to the first embodiment of the present invention.

FIG. 9 is a vertical cross-sectional view of the microneedle according to the first embodiment of the present invention.

FIG. 10 is a vertical cross-sectional view of the microneedle according to the first embodiment of the present invention.

FIGS. 11A, 11B and 11C are views sequentially showing a manufacturing process of the microneedle according to the first embodiment of the present invention, in which, FIG. 11A is a perspective view showing a state that a slide pin is inserted, FIG. 11B is a perspective view showing a state that a pair of slide cores is caused to draw closer to each other, and FIG. 11C is a perspective view showing a state that slide cores are caused to appear and abut on the slide pin.

FIGS. 12A, 12B, 12C and 12D are views sequentially showing the manufacturing process of the microneedle according to the first embodiment of the present invention, in which, FIG. 12A is a perspective view showing a state that resin is filled, FIG. 12B is a perspective view showing a state that the core pins are retracted, FIG. 12C is a perspective view showing a state that the slide pin is retracted, and FIG. 12D is a perspective view showing a state that the pair of slide cores is caused to separate from each other.

FIG. 13 is a perspective view of an essential part showing a state that the core pin is pressed against the slide pin and resin is filled, according to the first embodiment of the present invention.

FIG. 14 is an expanded view of a section XIV of FIG. 13 according to the first embodiment of the present invention.

FIG. 15 is a plan view of a microneedle according to a second embodiment of the present invention.

FIG. 16 is a side view of the microneedle according to the second embodiment of the present invention.

FIG. 17 is a plan view of a microneedle according to a third embodiment of the present invention.

FIG. 18 is a side view of the microneedle according to the third embodiment of the present invention.

FIG. 19 is a plan view of a microneedle according to a fourth embodiment of the present invention.

FIG. 20 is a side view of the microneedle according to the fourth embodiment of the present invention.

FIG. 21 is a plan view of a microneedle according to a fifth embodiment of the present invention.

FIG. 22 is a side view of the microneedle according to the fifth embodiment of the present invention.

FIG. 23 is a plan view of a microneedle according to a sixth embodiment of the present invention.

FIG. 24 is a side view of the microneedle according to the sixth embodiment of the present invention.

FIG. 25 is a front view of an upper part of a microneedle unit according to a seventh embodiment of the present invention.

FIGS. 26A and 26B are views according to the first embodiment of the present invention, in which, FIG. 26A is an electron microscope photograph showing a part of the microneedle unit, and FIG. 26B is an optical microscope photograph showing the microneedle.

FIGS. 27A and 27B are views according to the first embodiment of the present invention, in which, FIG. 27A is a photograph showing a state that the manufactured microneedle unit is attached to a lure lock syringe, and FIG. 27B is a microscope photograph showing a state that water is discharged uniformly from horizontal holes.

FIG. 28 is a perspective view explaining a manufacturing process for manufacturing microneedles having a multiple-raw configuration according to a eighth embodiment of the present invention.

FIG. 29 is a perspective view explaining the manufacturing process for manufacturing the microneedles having the multiple-raw configuration according to the eighth embodiment of the present invention.

FIG. 30 is a perspective view explaining the manufacturing process for manufacturing the microneedles having the multiple-raw configuration according to the eighth embodiment of the present invention.

FIG. 31 is a perspective view explaining the manufacturing process for manufacturing the microneedles having the multiple raw configuration according to the eighth embodiment of the present invention.

FIG. 32 is a perspective view explaining the manufacturing process for manufacturing the microneedles having the multiple-raw configuration according to the eighth embodiment of the present invention.

FIG. 33 is a perspective view explaining the manufacturing process for manufacturing the microneedles having the multiple raw configuration according to the eighth embodiment of the present invention.

FIG. 34 is a perspective view explaining the manufacturing process for manufacturing the microneedles having the multiple-raw configuration according to the eighth embodiment of the present invention.

FIG. 35 is a perspective view explaining the manufacturing process for manufacturing the microneedles having the multiple-raw configuration according to the eighth embodiment of the present invention.

FIG. 36 is a perspective view explaining the manufacturing process for manufacturing the microneedles having the multiple-raw configuration according to the eighth embodiment of the present invention.

FIG. 37 is a perspective view of a microneedle unit according to the eighth embodiment of the present invention.

FIG. 38 is a perspective view of a microneedle unit according to an ninth embodiment of the present invention.

FIG. 39 is a perspective view explaining relation between the microneedle unit and guide pins according to the ninth embodiment of the present invention.

FIG. 40 is a perspective view of a microneedle unit according to a tenth embodiment of the present invention.

FIG. 41 is a perspective view explaining relation between the microneedle unit and guide pins according to the tenth embodiment of the present invention.

### MODE(S) FOR CARRYING OUT THE INVENTION

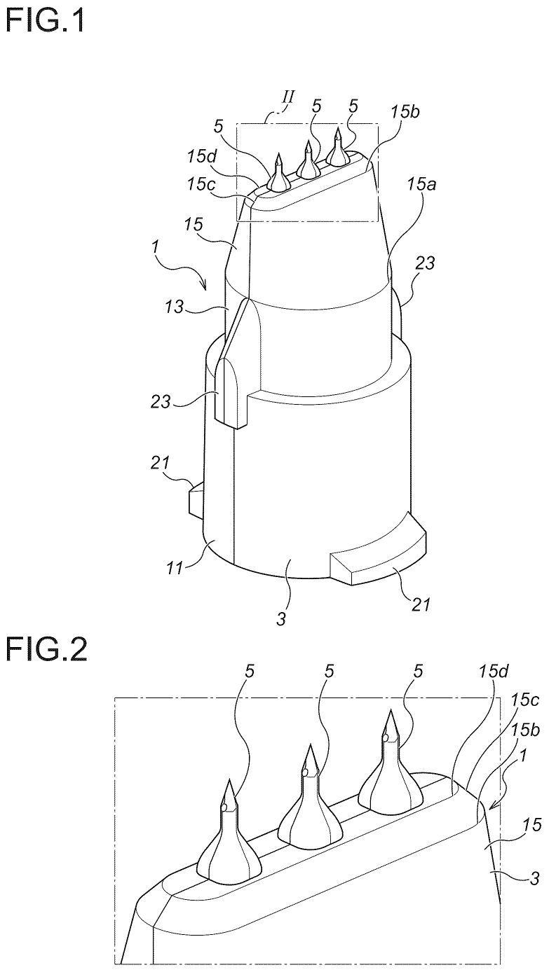

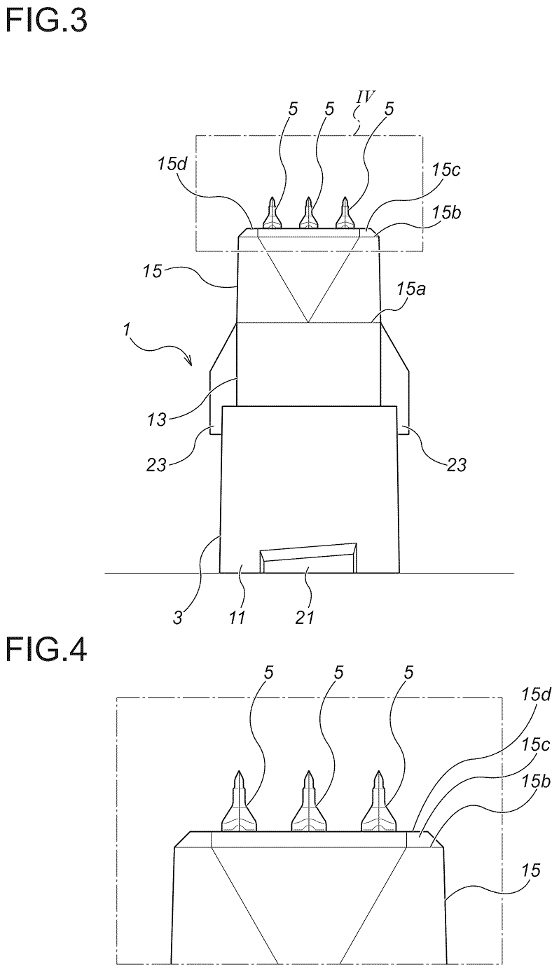

Now, a first embodiment of the present invention will be explained with reference to FIG. 1 and FIG. 14. FIG. 1 is a perspective view of a microneedle unit 1 according to a first embodiment of the present invention, and the microneedle unit 1 includes, a needle base 3, and a plurality of (in the present embodiment, three) microneedles 5 provided at the tip of the needle base 3. The microneedle unit 1 is made of resin, and of which whole body has been molded integrally.

The needle base 3 includes: a first cylindrical section 11; a second cylindrical section 13, integrally provided on a tip side of the first cylindrical section 11, and of which diameter is smaller than the first cylindrical section 11; and a microneedle pedestal 15 integrally provided on a tip side of the second cylindrical section 13. Flanges 21, 21 are provided, respectively, at positions opposing to each other at angle of 180 degrees, on a base end of the first cylindrical section 11. The flanges 21, 21 function to prevent disengagement when the microneedle unit 1 is coupled with an unillustrated screw in type of syringe (so-called “lure-lock type” of syringe). In a case where the syringe is not the screw-in type (so-called “lure-taper type”), the flanges 21, 21 are not necessary. An unillustrated hollow section is provided in the inside of the first cylindrical section 1, and a tip of an injection cylinder of the syringe is inserted into this hollow section detachably.

A pair of protrusions 23, 23 is protrusively provided respectively, extending from a lower part of the second cylindrical section 13 to an upper part of the first cylindrical section 11. The pair of the protrusions 23 is provided, at a position orthogonal to the pair of flanges 21, 21, and opposing to each other at angle of 180 degrees. An unillustrated cap covers the microneedle unit 1 on the side of the microneedles 5. When the cap is held to screw the microneedle unit 1 into the syringe, the protrusions 23, 23 function as rotation stoppers by abutting on ribs in the inside of the cap.

The microneedle pedestal 15 is in a taper shape, of which diameter decreases gradually toward the tip. Thus, with reference to the microneedle pedestal 15, the horizontal cross-sectional shape at a lower end 15a is in a circular shape, and the horizontal cross-sectional shape at an upper end 15b is in an oblong shape. The microneedle pedestal 15 is in a taper shape, connecting linearly between the circular-shaped lower end 15a and the oblong-shaped upper end 15b. There is an upper end surface 15d in an oblong shape, above the upper end 15b via a slanted surface 15c, and the three microneedles 5 described above are integrally provided on the upper end surface 15d.

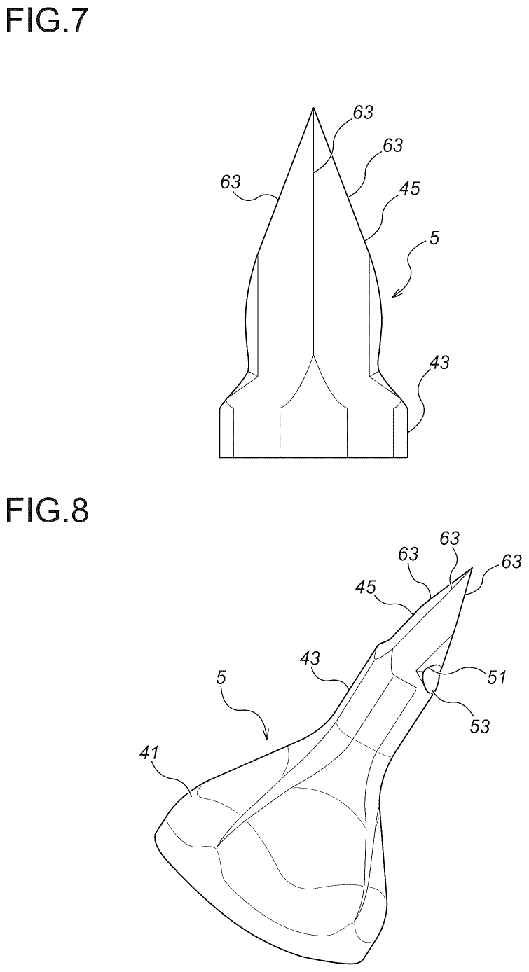

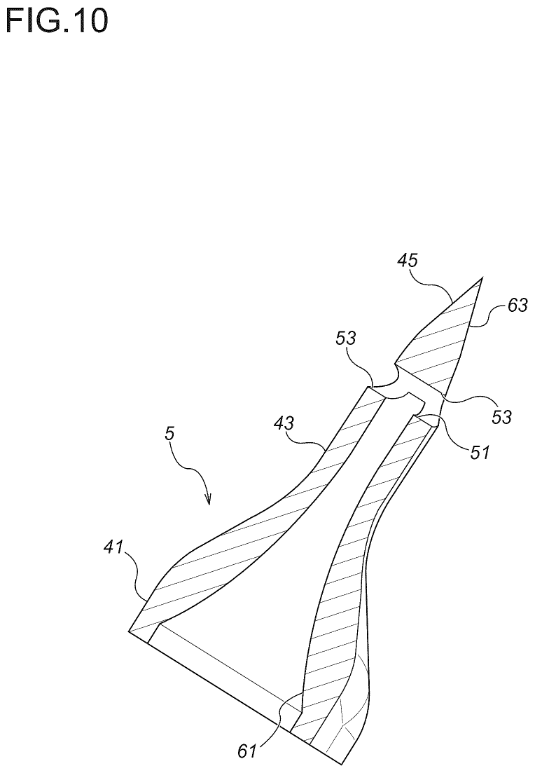

As illustrated in FIG. 7 to FIG. 10, the microneedle 5 includes: a foundation 41; a neck section 43 provided above the foundation 41; and a boring section 45 provided above the neck section 43. In the present embodiment, the boring section 45 is substantially in a pyramid shape.

With reference to the foundation 41, an area extending from the lowermost end to a predetermined height is in a square shape, of which four corners have been rounded respectively to form arcs, as seen by horizontal cross-sectional view. The shape is not required to be rectangle, and it is also possible to be in a circular shape, etc. A diameter decreases to form a taper shape, from an uppermost position of the area of the predetermined height, to the lowermost end of the neck section 43. The horizontal cross-section of the neck section 43 is in a square shape, of which four corners have been rounded respectively to form arcs, and this square is shifted by 45 degrees against the square of the foundation 41. A horizontal hole 51 is provided in a lower part of the boring section 45, and the horizontal hole 51 is penetrated, of which both openings serve as medical solution discharge ports 53, 53.

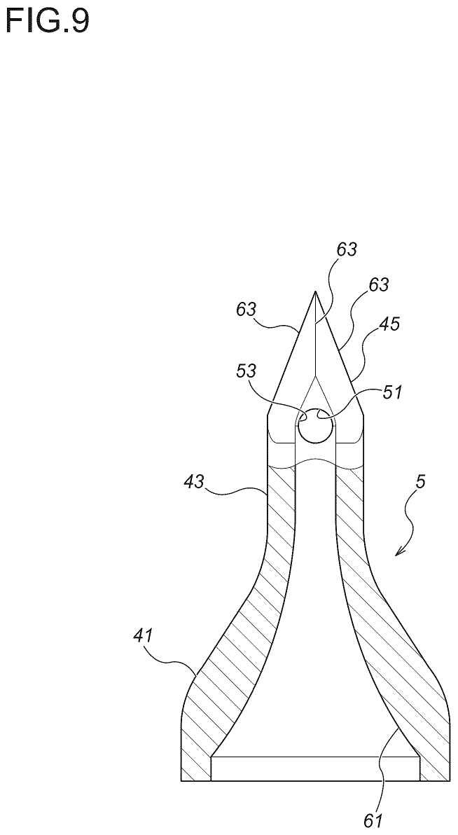

As illustrated in FIG. 9, a vertical hole 61, serving as a medical solution flow path, is formed in the inside of the foundation 41 and the neck section 43, and the vertical hole 61 is formed to become narrower gradually in an area extending from the foundation 41 to the neck section 43.

The boring section 45 is in a square shape as seen by horizontal cross-sectional view, and edges 63, 63, 63, 63 are provided along four directions orthogonal to each other. The horizontal hole 51, described above, is provided along a ridgeline of the boring section 45, that is, along a line connecting between a pair of edges 63, 63 opposing to each other, among the four edges 63, 63, 63, 63, and the medical solution discharge ports 53, 53 are provided at the both ends of the ridgeline. Moreover, the lower part of each of the edges 63, 63, 63, 63 is formed in an arc shape protruding outwardly.

The length of the microneedle 5 is preferably 0.2 to 2.0 mm from viewpoints of puncturing performance, molding facility, medical agent diffusion property, etc., and more preferably, 0.5 to 1.5 mm. Moreover, the thickness of a needle section (the thickness of the neck section 43) of the microneedle 5 is preferably 0.05 to 0.3 mm, from viewpoints of strength, damage to skin, molding facility, etc., and more preferably, 0.1 to 0.25 mm. Moreover, with reference to the size of the vertical hole 61 and the horizontal hole 51, the diameter thereof is preferably 0.03 to 0.2 mm, and more preferably, 0.05 to 0.1 mm. Moreover, the interval between each of the microneedles 5 is preferably 0.5 to 3.0 mm.

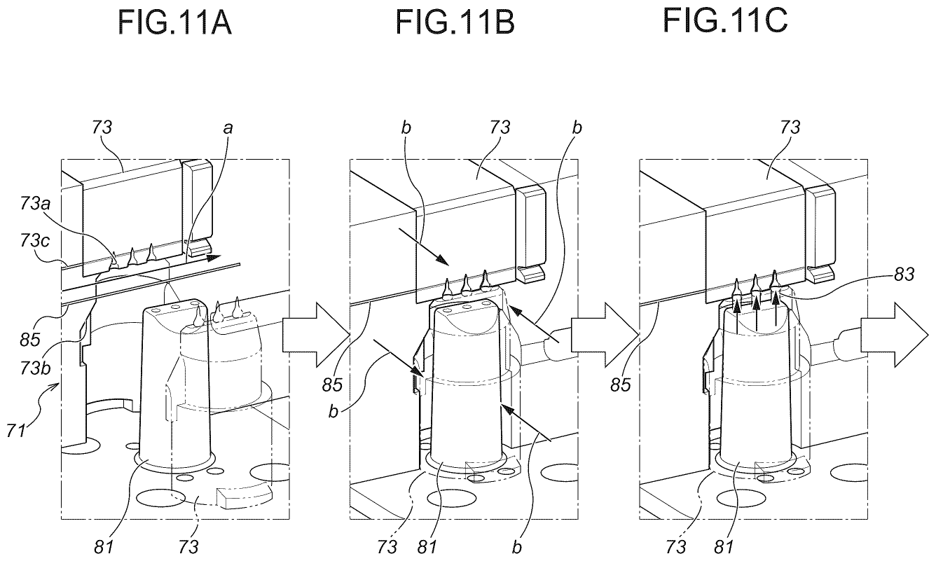

The microneedle unit 1 having the above configuration is manufactured by a microneedle manufacturing machine 71 as illustrated in FIGS. 11A and 11C. As illustrated in FIG. 11A, the microneedle manufacturing machine 71 includes a pair of slide cores 73, 73 arranged to be joinable to and separatable from each other in a horizontal direction orthogonal to an axis direction of the microneedle 5, and is also provided with an unillustrated slide pin driving mechanism, and other mechanical units. The slide cores 73, 73 are arranged in a size allowing integral molding of the needle base 3 and the microneedles 5.

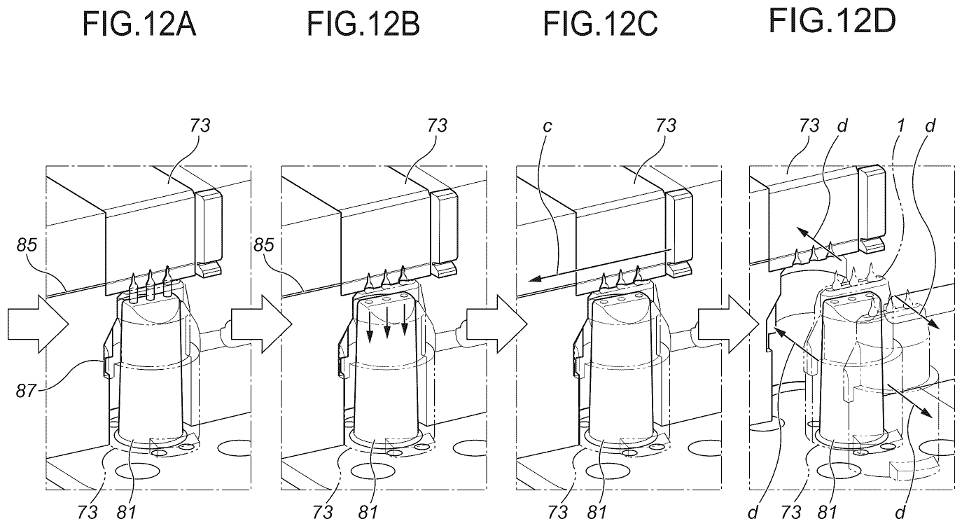

Note that, in FIGS. 11A, 11B, 11C and FIGS. 12A, 12B, 12C, 12D, the slide core 73 on one side is shown by solid lines, and the slide core 73 on the other side is shown by imaginary lines.

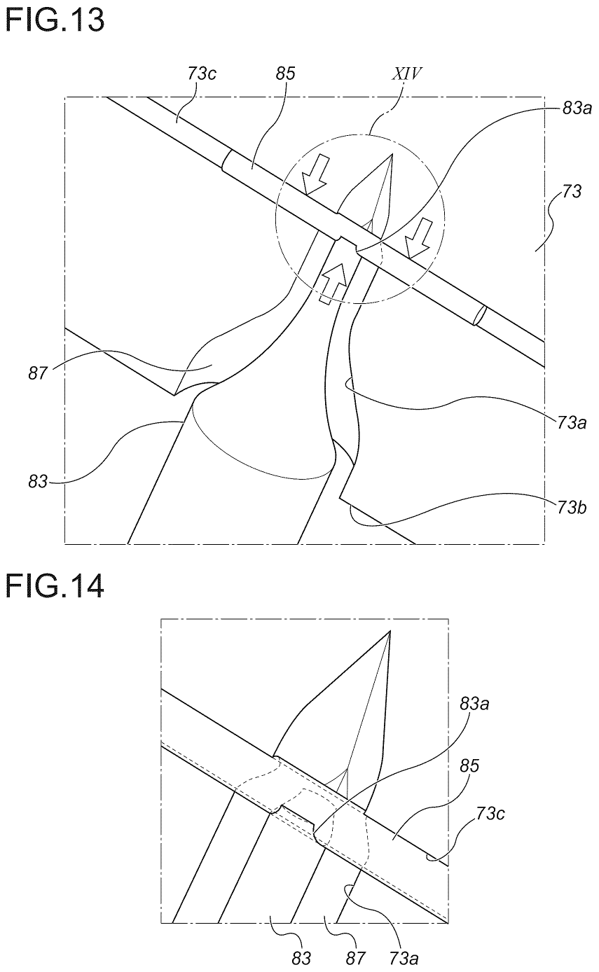

As illustrated in FIG. 11A, a core insert 81 is arranged between the pair of slide cores 73, 73, and a plurality of (in the present embodiment, three) core pins 83, 83, 83 is provided to be protrusible from and retractable into an upper end of the core insert 81. As illustrated in FIG. 13 and FIG. 14, an engagement recess 83a is formed at an upper end of the core pin 83, and the cross-section of the engagement recess 83a is in a semicircular shape. The pair of slide cores 73, 73 is arranged to be joinable to and separatable from each other, by sandwiching the core insert 81. The core insert 81 is also arranged in a size allowing integral molding of the needle base 3 and the microneedles 5.

As illustrated in FIG. 11A, the slide core 73 is provided with a cavity 73a to mold the microneedles 5, and a cavity 73b to mold the needle base 3. A slide pin 85 is arranged between the pair of slide cores 73, 73 above the core insert 81, to be protrusible and retractable by the slide pin driving mechanism. Recesses 73c, 73c, for engaging with the slide pin 85, are formed respectively in the slide cores 73, 73. The cross section of the slide pin 85 is in a circular shape.

Now, a microneedle manufacturing method by using the microneedle manufacturing machine 71 will be explained sequentially.

First, as illustrated in FIG. 11A, the pair of slide cores 73, 73 is separate from each other, and the three core pins 83, 83, 83 are retracted in the core insert 81. Next, as shown by an arrow a, the slide pin 85 appears between the pair of slide cores 73, 73 by the slide pin driving mechanism.

Next, as shown by an arrow b of FIG. 11B, the pair of slide cores 73, 73 is caused to move closer to each other, and consequently, to sandwich the slide pin 85, and to surround the core insert 81 with remaining a clearance therefrom. Thus, the slide pin 85 is held by the recesses 73b, 73b of the pair of slide cores 73, 73, and consequently, the rigidity of the slide pin 85 is secured, and the deflection thereof is prevented. Moreover, the core insert 81 is sandwiched by the cavities 73b, 73b of the pair of slide cores 73, 73, with remaining the clearance at a predetermined distance.

Next, as illustrated in FIG. 11C, the three core pins 83, 83, 83 appear upwardly from the core insert 81. The engagement recesses 83a, 83a, 83a, respectively at the tip of the appearing core pins 83, 83, 83, engage with the slide pin 85. Thus, the positioning of each of the core pins 83, 83, 83 is performed, and because of application of pressure during filling of resin, which will be described afterwards, the slide pin 85 is prevented from improper positioning or deformation. Similarly, because of abutting on the slide pin 85, the core pins 83, 83, 83 are also prevented from improper positioning or deformation.

Next, as illustrated in FIG. 12A, after a mold is closed completely, resin is injected from a molding machine. First, resin 87 flows into the clearance between the cavity 73b and the core insert 81. Next, the resin 87 flows toward the tip, and by flowing into the clearance between the core pins 83, 83, 83 and the cavity 73a, the vertical hole 61 is formed. Thereafter, the resin 87 flows into the clearance between the slide pin 85 and the cavity 73a, and consequently, the horizontal hole 51 is formed, and eventually, the resin reaches the tip and the injection operation is finished.

Next, after passing of a predetermined curing time (period) for cooling, as illustrated in FIG. 12B, the core pins 83, 83, 83 are retracted into the core insert 81.

Next, as shown by an arrow c of FIG. 12C, the slide pin 85 is retracted in the slide pin driving mechanism.

Next, as shown by an arrow d of FIG. 12D, the pair of slide cores 73, 73 is caused to move to separate from each other, and the microneedle unit 1 as a molded product according to the present embodiment, is taken out.

The filling process of resin as illustrated in FIG. 12A will be explained in details. FIG. 13 is an expanded cross-sectional view showing the filling process of resin, and FIG. 14 is a further expanded cross-sectional view of a section XIV of the FIG. 13. The slide pin 85 is held by the recesses 73c, 73c of the pair of slide cores 73, 73, and accordingly, the rigidity of the slide pin 85 is secured. In such a state, pressure is applied to the resin 87 at a predetermined molding pressure, and the resin 87 is filled. At that time, because the core pin 83 is pressed against the slide pin 85, it is possible to resist the molding pressure, and improper positioning as well as deformation of both the core pin 83 and the slide pin 85 are prevented. Consequently, it is possible to suppress generation of burrs at an abutting part between the slide pin 85 and the core pin 83.

Note that, in the present embodiment, the upper part of the core pin 83 is formed to serve as the engagement recess 83a, of which cross section is in a semicircular shape to coincide with an outer diameter of the slide pin 85. However, the present embodiment is not limited to such a configuration, and any modification is possible, such as engagement of V-shaped or flat-shaped profiles, or other shapes. Moreover, it is also possible to improve close contact property by plating or coating on a surface of the pin.

The present embodiment described above has the following effects.

First, it is possible to manufacture the microneedle 5 integrally and easily, provided with the vertical hole 61 serving as the medical solution flow path, and also with the horizontal hole 51 connected thereto and serving as the medical solution discharge ports, in the penetrated state. This is because, the pair of slide cores 73, 73 is arranged to be joinable and separatable, and the core insert 81 is arranged therebetween, provided with the core pins 83 to be protrusible from and retractable in the core insert 81, and furthermore, the slide pin 85 is arranged to be protrusible and retractable for forming the horizontal hole 51.

Moreover, because the slide pin 85 is held by the recesses 73c, 73c of the pair of the slide cores 73, 73, the rigidity thereof is secured, and during application of pressure for filling the resin 87, the slide pin 85 is prevented from improper positioning or deformation, and it is possible to form the horizontal hole 51 precisely, and furthermore, it is possible to eliminate generation of burrs.

In particular, because the both sides of the recesses 73b immediately adjacent to each other, for forming the microneedles 5, are held strongly at a considerably short distance like a both ends supported beam, the rigidity improves still further.

Moreover, because the core pin 83 is caused to become close contact with the slide pin 85, this configuration also functions to prevent improper positioning or deformation of the slide pin 85, and it is possible to mold the microneedle 5 precisely.

In particular, because the cross section of the engagement recess 83a of the core pin 83 is in a semicircular shape, to coincide with an outer circumference of the circular-shaped cross section of the slide pin 85, the above effects are further enhanced.

Moreover, because the position of the core pin 83 in itself is also maintained, the core pin 83 is prevented from improper positioning or deformation during molding, and it is possible to mold the microneedle 5 precisely.

In particular, the engagement recess 83a, of which cross section is in a semicircular shape, is provided at the tip of the core pin 83, and because the engagement recess 83a coincide with the outer shape of the slide pin 85, the above effects are further enhanced.

Moreover, because the pair of slide cores 73, 73 is set to be in a size allowing integral molding of the microneedles 5 and the needle base 3, and also because the core insert 81 is also set to be in a size allowing integral molding of the microneedles 5 and the needle base 3, it is possible to mold the microneedle unit 1 easily, in which the microneedles 5 and the needle base 3 have been integrally formed. Therefore, it is not necessary to prepare the needle base 3 as a separate component.

Moreover, because of integral molding, the airtightness is perfect as compared with a case of the separate component.

Moreover, because the microneedle 5 is not formed by bonding, but formed integrally, the mechanical strength is excellent.

Moreover, as compared with conventional bonding methods, the airtightness of the medical solution flow path is secured perfectly.

Moreover, because the pyramid-shaped boring section 45 is provided at the tip, and because the four edges 63 are provided, it is possible to perform puncture by sharply cutting the skin.

Moreover, because the lower part of the edge 63 is in a round shape, when withdrawing from the skin after puncturing, no damage is given to the skin.

Moreover, because the cross section of the horizontal hole 51 is in a circular shape, the resistance when injecting of medical solution is small.

Moreover, because the neck section 43 is provided below the boring section 45, and the neck section 43 becomes close contact with the skin during puncturing, it is possible to prevent leaking of injected medical solution.

Moreover, because the foundation 41 is provided below the neck section 43, and the connecting section therebetween is in an arc shape, it is possible to improve the mechanical strength of the microneedle 5.

Moreover, because the foundation 41 stretches the skin during puncturing, the puncture can be performed easily, and it is possible to unify the puncturing depth.

Moreover, because the vertical hole 61 is formed to become gradually narrower from the lower side toward the upper side, it is possible to perform injection of medical solution efficiently.

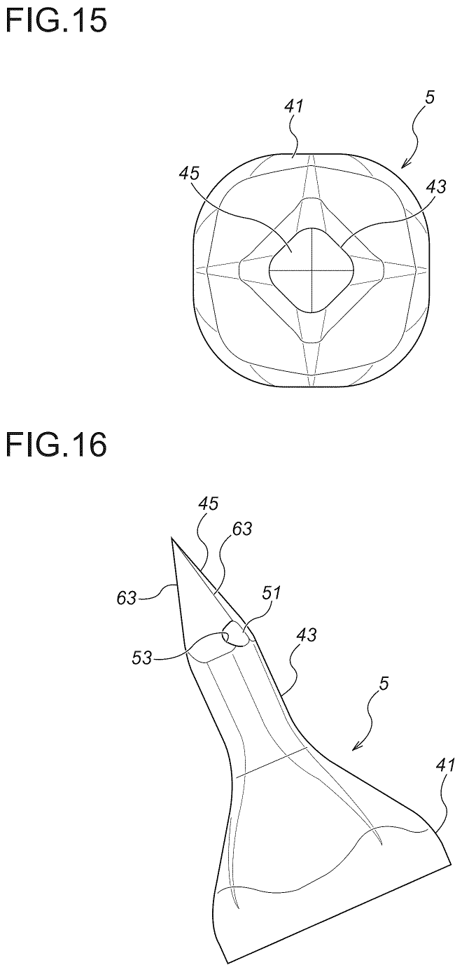

Next, a second embodiment of the present invention will be explained with reference to FIG. 15 and FIG. 16. In the second embodiment, the round shape at the lower part of the edge 63 of the pyramid-shaped boring section 45 is omitted.

The other configurations are substantially the same as those of the first embodiment, and therefore, the same reference signs will be allotted to the same elements in the drawings, and the explanation thereof will be omitted.

Accordingly, it is possible to achieve the same effects as those of the first embodiment.

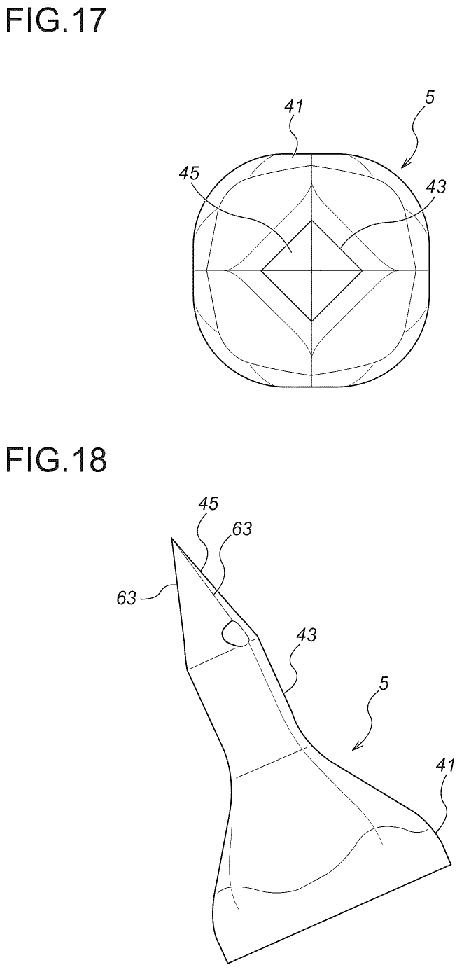

Next, a third embodiment of the present invention will be explained with reference to FIG. 17 and FIG. 18. In the third embodiment, with reference to the second embodiment, the horizontal cross section of the neck section 43 is in a square shape.

The other configurations are substantially the same as those of the second embodiment, and therefore, the same reference signs will be allotted to the same elements in the drawings, and the explanation thereof will be omitted.

Accordingly, it is possible to achieve the same effects as those of the second embodiment.

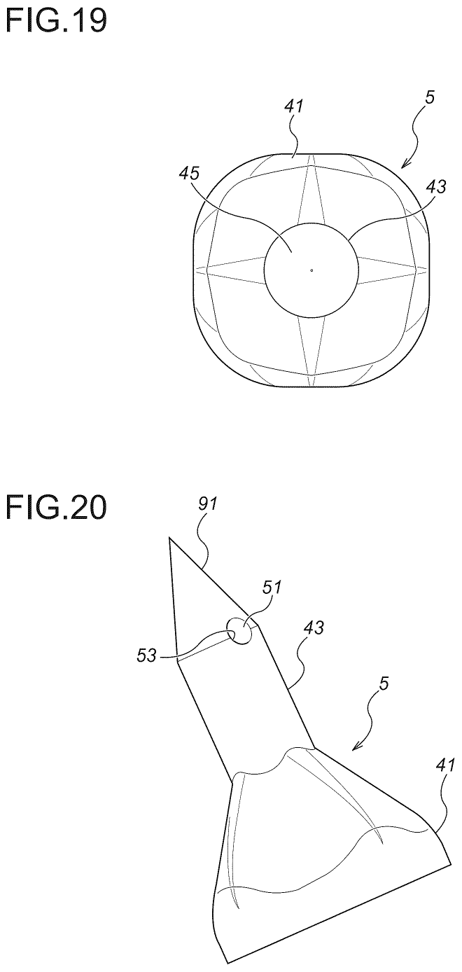

Next, a fourth embodiment of the present invention will be explained with reference to FIG. 19 and FIG. 20. In the fourth embodiment, a cone section 91 is provided at the tip, and the horizontal cross section of the neck section 43 is in a circular shape.

The other configurations are substantially the same as those of the first embodiment, and therefore, the same reference signs will be allotted to the same elements in the drawings, and the explanation thereof will be omitted.

Accordingly, it is possible to achieve the same effects as those of the first embodiment.

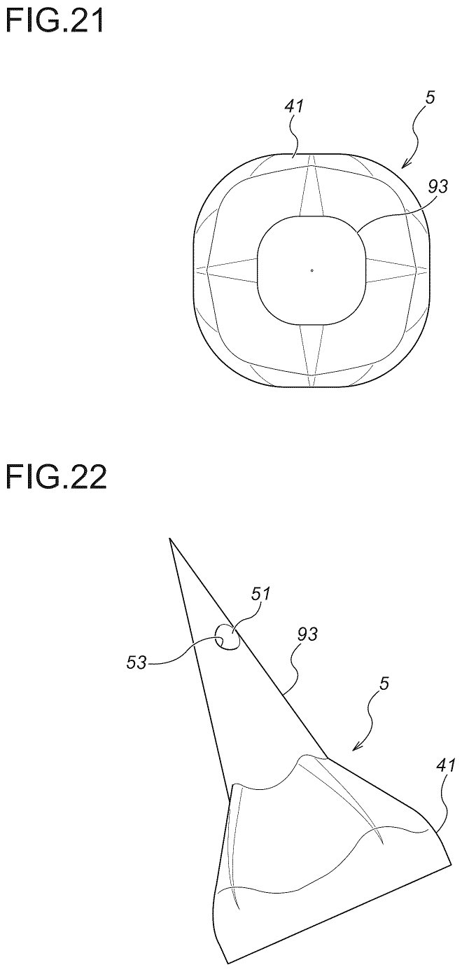

Next, a fifth embodiment of the present invention will be explained with reference to FIG. 21 and FIG. 22. In the fifth embodiment, a cone section 93 is provided, instead of the boring section and the neck section of the first embodiment.

The other configurations are substantially the same as those of the first embodiment, and therefore, the same reference signs will be allotted to the same elements in the drawings, and the explanation thereof will be omitted.

Accordingly, it is possible to achieve the same effects as those of the first embodiment.

Moreover, because the tip is thinner and sharper, the puncture can be performed easily, and the damage to the skin is reduced.

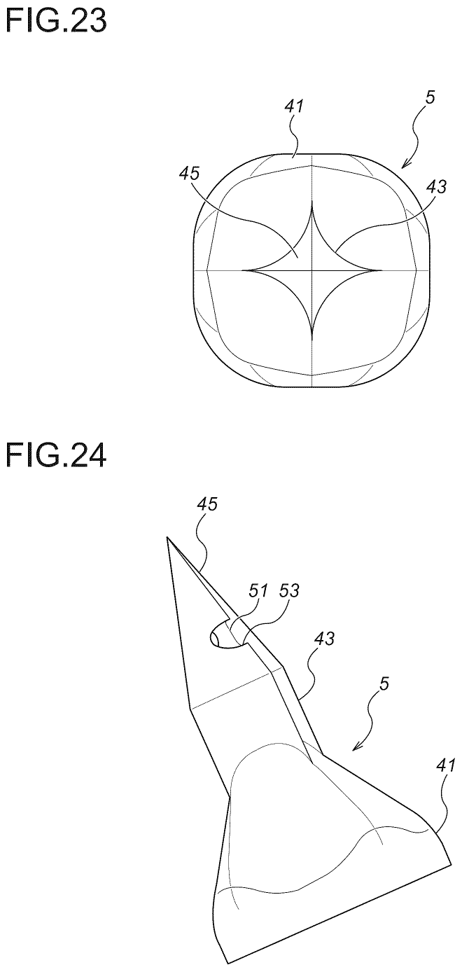

Next, a sixth embodiment of the present invention will be explained with reference to FIG. 23 and FIG. 24. In the sixth embodiment, each of the side surfaces of the pyramid-shaped boring section 45 of the first embodiment is formed in a recessed shape, and each of the side surfaces of the neck section 43 is also formed in a recessed shape.

The other configurations are substantially the same as those of the first embodiment, and therefore, the same reference signs will be allotted to the same elements in the drawings, and the explanation thereof will be omitted.

Accordingly, it is possible to achieve the same effects as those of the first embodiment.

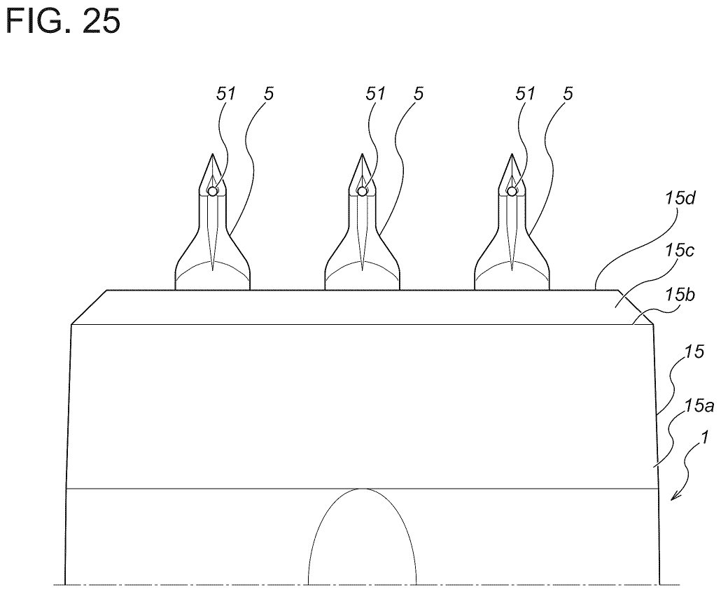

Next, a seventh embodiment of the present invention will be explained with reference to FIG. 25. In the first to sixth embodiments, the horizontal holes 51 are formed linearly through the three microneedles 5, 5, 5, by using a single slide pin 85. In contrast, in the seventh embodiment, as illustrated in FIG. 25, the horizontal holes 51 are formed in the three microneedles 5, 5, 5, in directions parallel to each other. Thus, a single slide pin 85 and a single core pin 83 are arranged for each of the three microneedles 5, 5, 5 (in total, three slide pins 85 and three core pins 83), and the same operations as those of the first embodiment are performed for each of the microneedles 5.

The other configurations are substantially the same as those of the first to sixth embodiments, and therefore, the same reference signs will be allotted to the same elements in the drawings, and the explanation thereof will be omitted.

Accordingly, it is possible to achieve the same effects as those of the first to sixth embodiments.

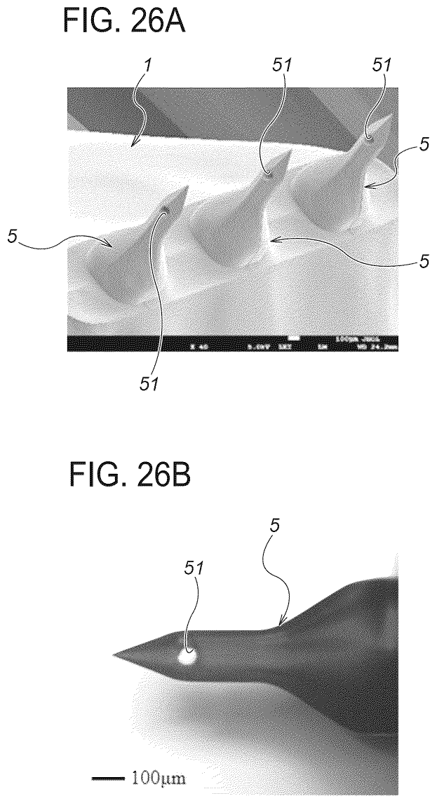

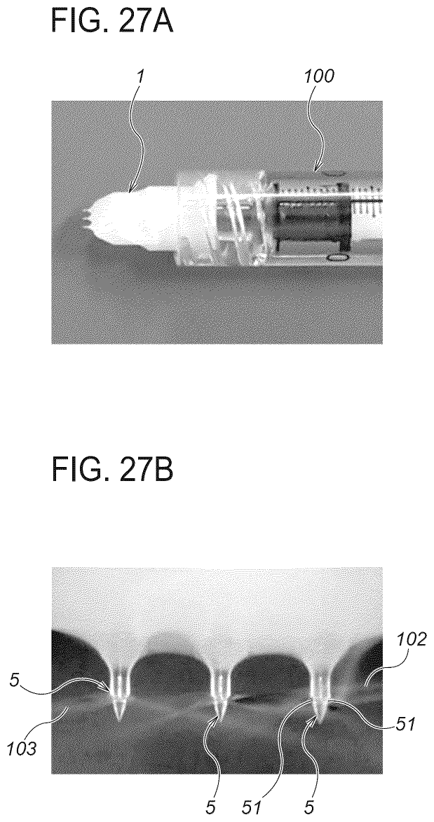

Next, FIGS. 26A and 26B and FIGS. 27A and 27B show the microneedle unit 1 actually manufactured according to the first embodiment.

FIG. 26A is an electron microscope photograph showing a part of the three microneedles 5, 5, 5 of the microneedle unit 1 actually manufactured, and FIG. 26B is an optical microscope photograph showing a single microneedle 5.

FIG. 27A is a photograph showing a state that the manufactured microneedle unit 1 is attached to a lure locksyringe 100, and FIG. 27B is a microscope photograph showing a state that water 102 is discharged uniformly from the horizontal holes 51.

The present example was manufactured under molding conditions that, the material was polyglycol acid (PGA), mold temperature was 120° C.,resin temperature was 240° C., resin pressure was 110 MPa, clamping force was 10 t, and molding cycle was approximately 40 seconds.

Thus, the microneedle unit 1 provided with the horizontal hole 51, clearly penetrating therethrough at φ80 μm, was manufactured integrally and successfully. The tip and the pyramid-shaped edges of the microneedle 5 were formed sharply. Moreover, the needle intervals were 1.3 mm, the needle length (effective puncture depth) was 0.65 mm, and the needle width was 0.22 mm.

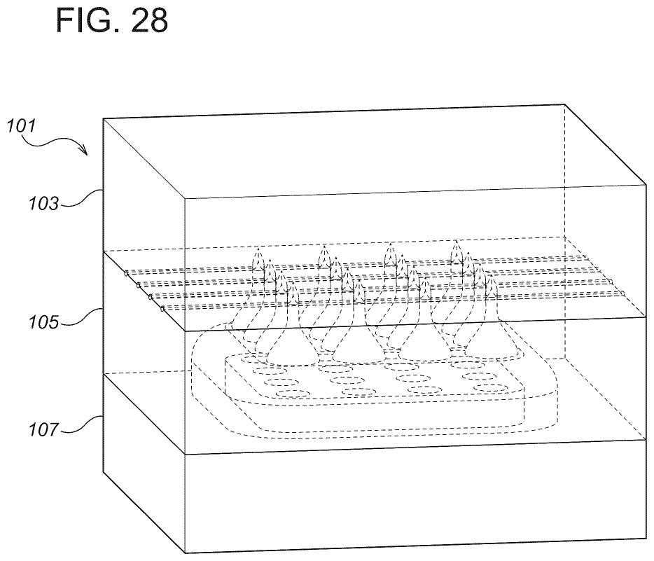

Next, an eighth embodiment of the present embodiment will be explained with reference to FIG. 28 to FIG. 37. The first embodiment has been explained with regard to a case of manufacturing of the microneedle unit 1 provided with a plurality of (in the first embodiment, three) microneedles 5 arranged in a single row. In contrast, the eighth embodiment will be explained with regard to a case of manufacturing of a microneedle unit 1′ provided with the microneedles 5 in multiple rows.

Moreover, in the first embodiment, with reference to the microneedle manufacturing machine 71, the pair of slide cores 73, 73 is arranged to be joinable and separatable in the direction (in the first embodiment, in the horizontal direction) orthogonal to the axis direction of the microneedle. In contrast, the eighth embodiment will be explained in regard to an example, in which, with reference to a microneedle manufacturing machine 101, a cavity insert upper plate 103 and a cavity insert lower plate 105, corresponding to the pair of slide cores, are arranged to be joinable and separatable in the axis direction (in the present embodiment, vertical direction) of the microneedles, and a core insert 107 is arranged to be ascendable and descendable in the vertical direction below the cavity insert lower plate 105. Now, the configuration will be explained in details.

In the cavity insert upper plate 103, a plurality of (in the present embodiment, four pieces by four rows, in total sixteen) recesses 109 for manufacturing tips of microneedles has been formed, and furthermore, a plurality of (in the present embodiment, four) slide pin accommodating recesses 111 has also been formed. The cross section of the slide pin accommodating recess 111 is in a semicircular shape facing downward.

In the cavity insert lower plate 105, a cavity 113 has been formed, and furthermore, a plurality of (in the present embodiment, four) slide pin accommodating recesses 115 has also been formed. The cavity 113 includes: a base cavity 113a corresponding to a base 3′ of the microneedle unit 1′; and sixteen microneedle cavities 113b corresponding to the microneedles 5, which are four pieces by four rows, in total sixteen. The cross section of the slide pin accommodating recess 115 is in a semicircular shape facing upward.

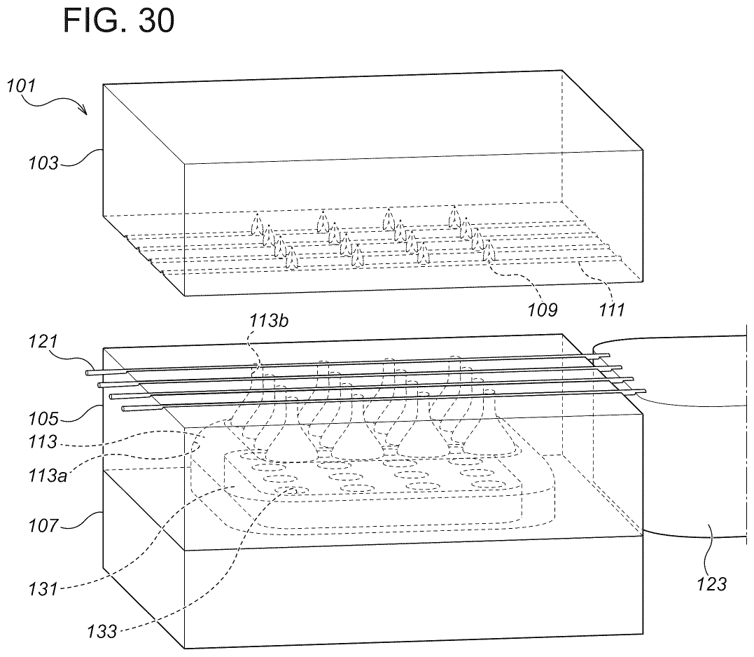

As illustrated in FIG. 30, four slide pins 121 are arranged to be protrusible and retractable in the horizontal direction, and the four slide pins 121 are sandwiched by the respective slide pin accommodating recesses 111, 115. The slide pins 121 are driven by a slide pin driving unit 123.

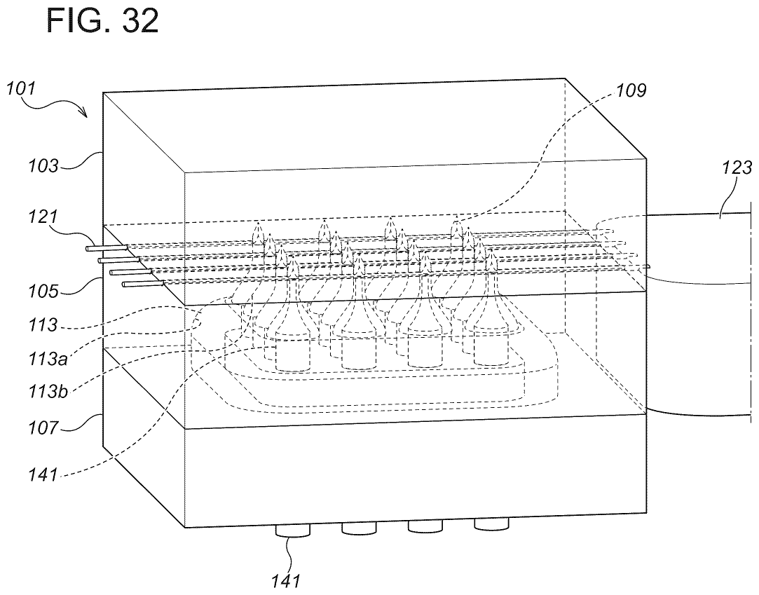

Aprotrusion 131 is protrusively provided for forming the base 3′ of the microneedle unit 1′, and sixteen through holes 133, corresponding to the microneedles 5, which are four pieces by four rows, in total sixteen, are penetrating through the protrusion 131. As illustrated in FIG. 32, a core pin 141 is arranged to be ascendable and descendable by penetrating through each of the sixteen through holes 133.

Now, the manufacturing process will be explained sequentially.

FIG. 28 illustrates a state before operation, in which, the slide pins 121 and the core pins 141 are not inserted, and resin is not filled.

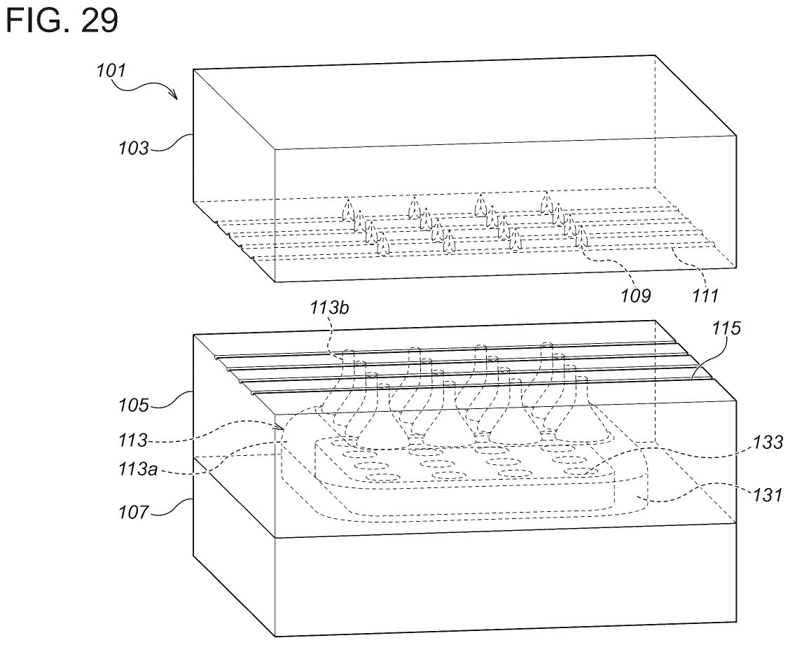

Next, as illustrated in FIG. 29, the cavity insert upper plate 103 is caused to move upwardly, in order to insert the slide pins 121.

Next, as illustrated in FIG. 30, the slide pins 121 are inserted into spaces between the cavity insert upper plate 103 and the cavity insert lower plate 105.

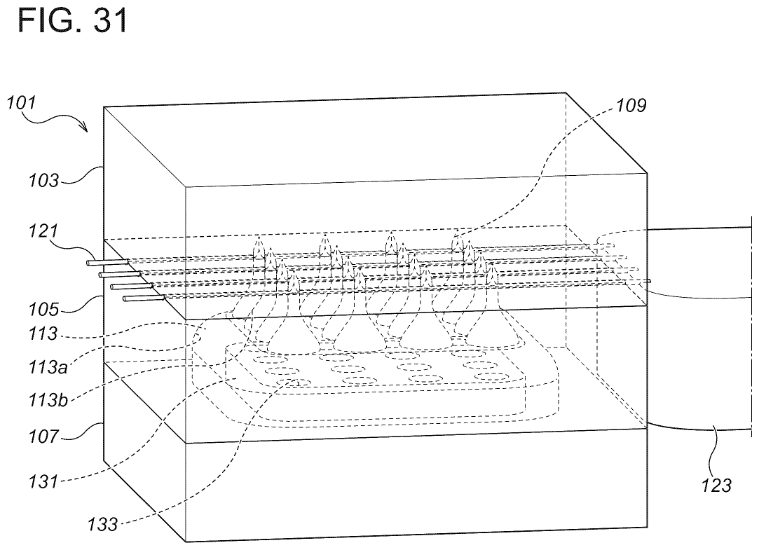

Next, as illustrated in FIG. 31, the cavity insert upper plate 103 is caused to move downwardly, and consequently, the slide pins 121 are sandwiched by the cavity insert upper plate 103 and the cavity insert lower plate 105.

Next, as illustrated in FIG. 32, the core pins 141 are caused to move upwardly, and consequently, an engagement recess at the tip of the core pins 141 engages with the slide pin 121. Thus, the positioning of the slide pin 121 is performed, and the slide pin 121 is prevented from improper positioning or deformation due to application of pressure during filling of resin.

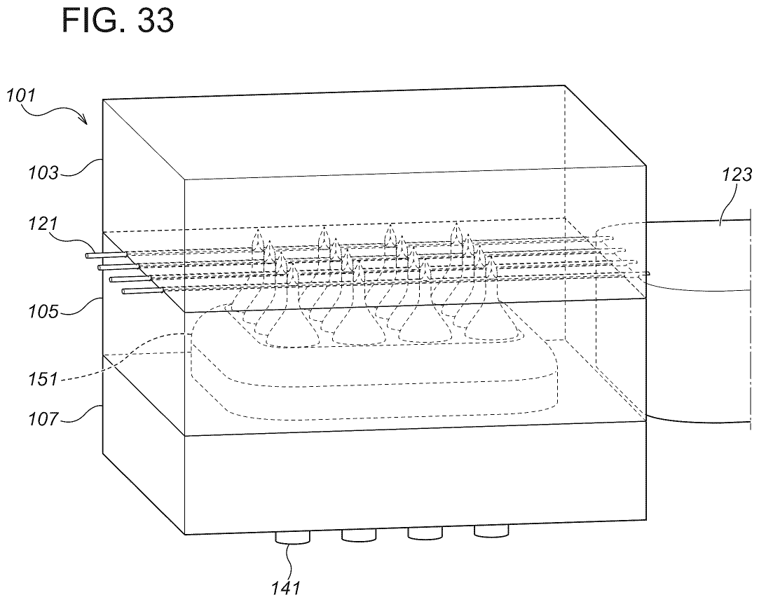

Next, as illustrated in FIG. 33, resin 151 is injected, and the resin 151 is filled, into the inside of a cavity 109 of the cavity insert upper plate 103, into a clearance between the cavity 113 of the cavity insert lower plate 105 and the protrusion 131 of the core insert 107, and into a clearance between the cavity 113 of the cavity insert lower plate 103 and the core pins 141.

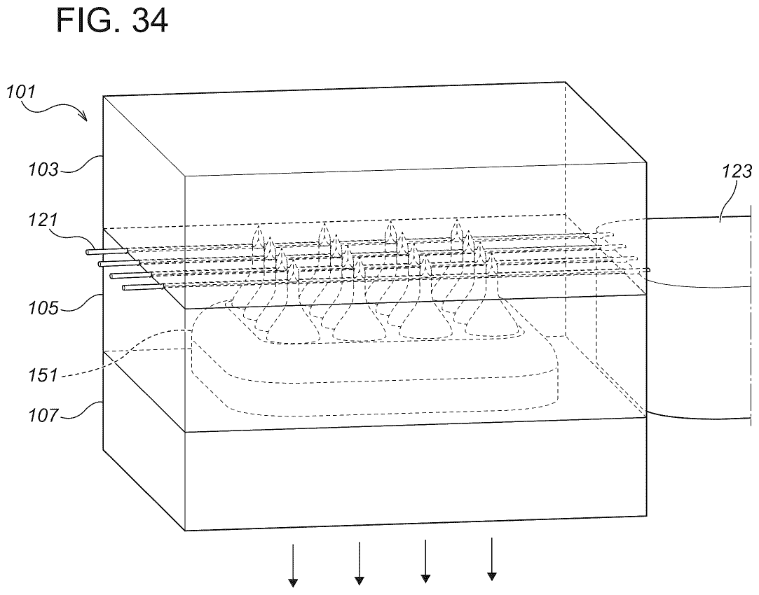

Next, as illustrated in FIG. 34, after passing of a predetermined curing period, the core pins 141 are caused to move rearwardly.

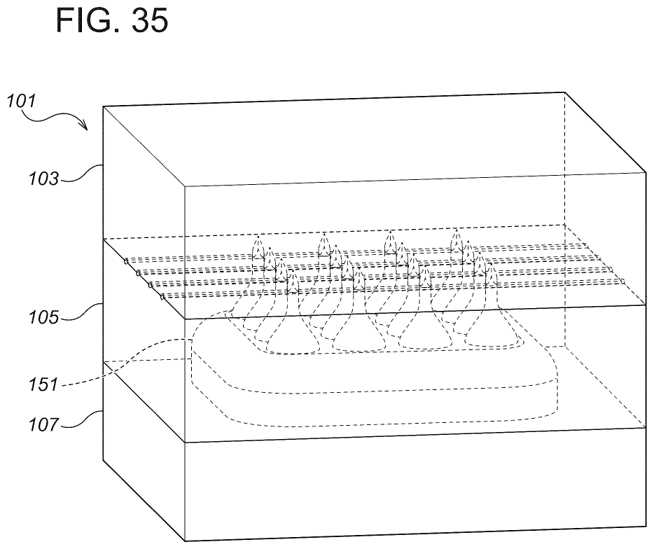

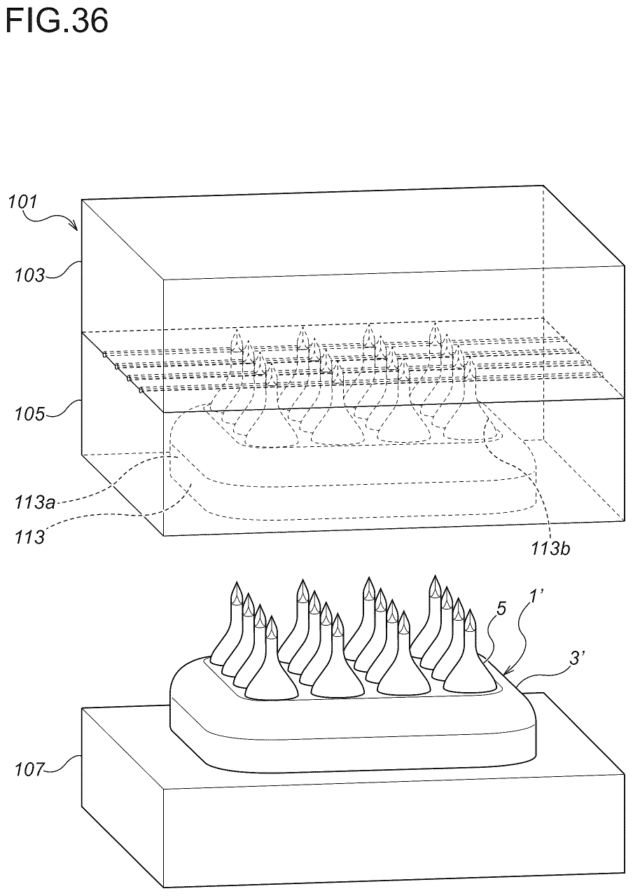

Next, as illustrated in FIG. 35, the slide pins 121 are caused to move rearwardly.

Next, as illustrated in FIG. 36, the core insert 107 is caused to move downwardly, and the molded microneedle unit 1′ is exposed and taken out.

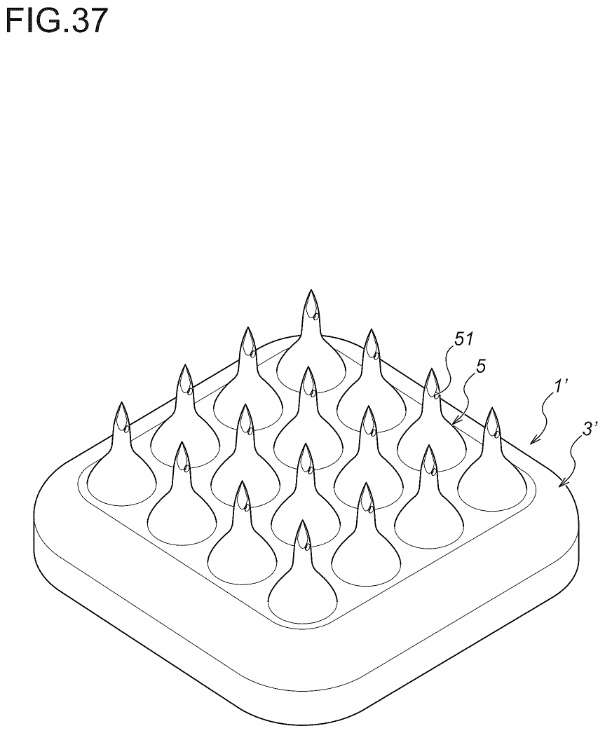

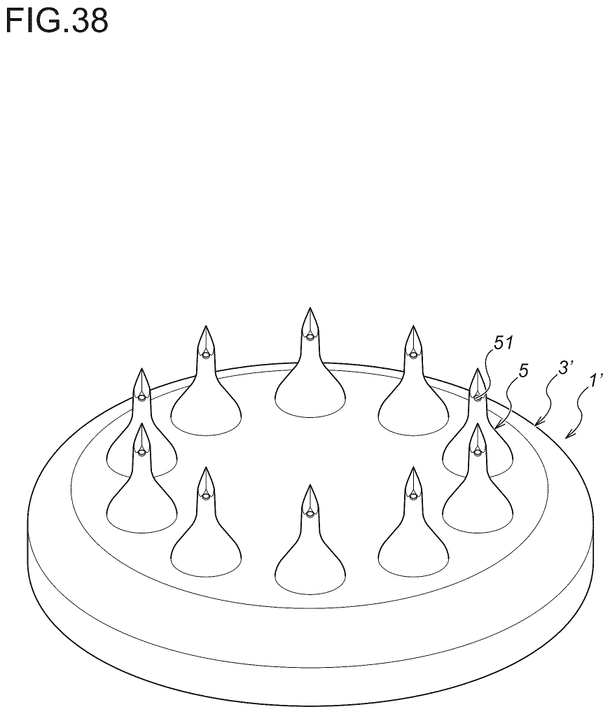

The microneedle unit 1′ has a configuration as illustrated in FIG. 37.

Accordingly, it is possible to achieve the same effects as those of the first to seventh embodiments.

Moreover, by using this multiple-row type of microneedle unit 1′, it is possible to inject medical solution or collect interstitial fluid, in a wider area.

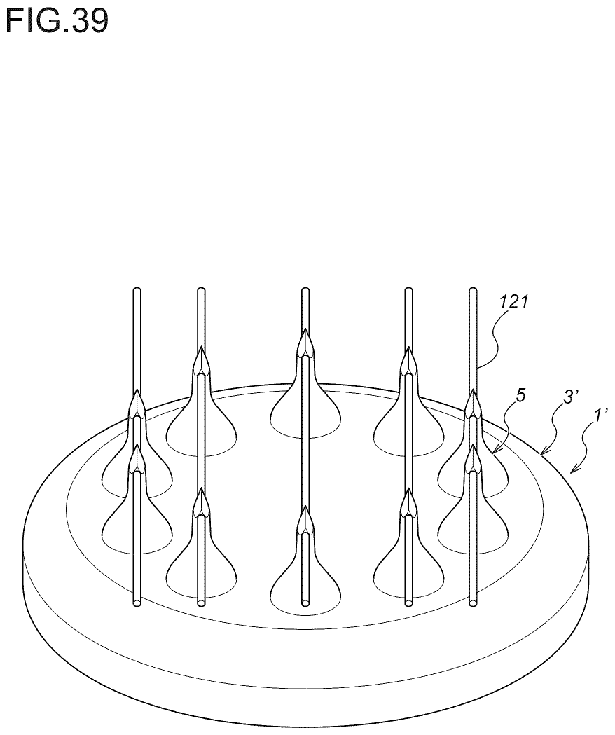

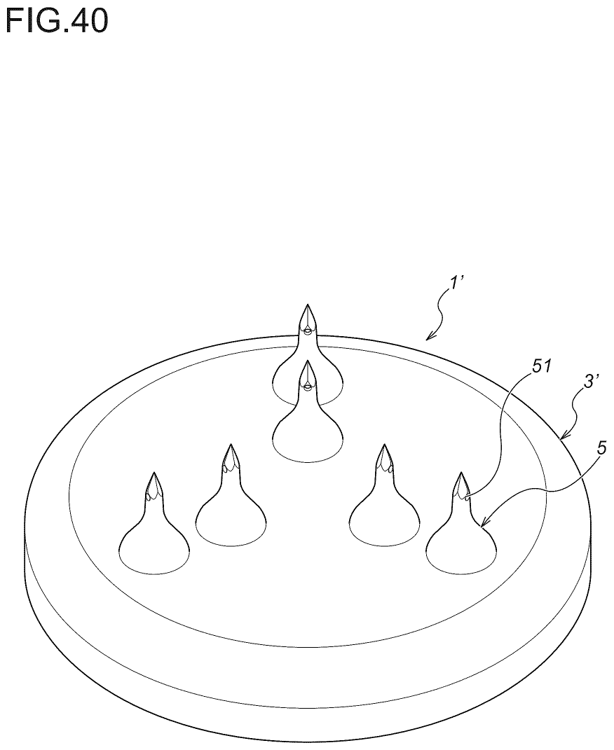

Next, a ninth embodiment of the present invention will be explained with reference to FIG. 38 and FIG. 39. In the ninth embodiment, thebase 3′ of the microneedle unit 1′ is in a circular shape as seen by plan view, and a plurality of (in the present embodiment, ten) microneedles 5 is arranged circumferentially. The horizontal hole 51 of each of the microneedles 5 is oriented in the same direction, and as illustrated in FIG. 39, and the microneedles 5 are formed by five slide pins 121.

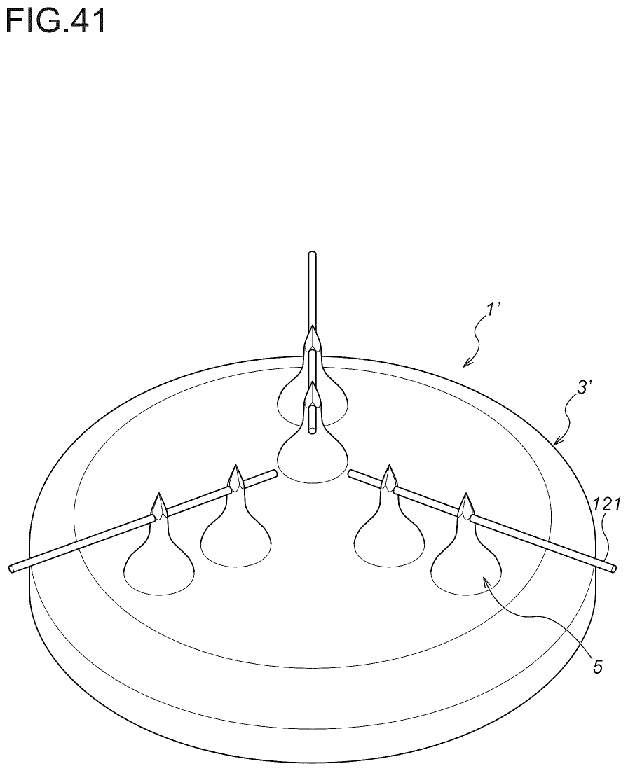

Next, a tenth embodiment of the present invention will be explained with reference to FIG. 40 and FIG. 41. In the tenth embodiment, the base 3′ of the microneedle unit 1′ is in a circular shape as seen by plan view, and a plurality of (in the present embodiment, six) microneedles 5 is arranged, two by two in radial directions, at positions with angle of 120 degrees. The horizontal hole 51 of each of the microneedles 5 is oriented in the radial direction, and as illustrated in FIG. 41, and the microneedles 5 are formed by three slide pins 121.

Note that, the present invention is not limited to the first to tenth embodiments described above.

First, the first to seventh embodiments are explained for the cases of integral molding of the whole body extending from the microneedles to the needle base, and the eighth to tenth embodiments are explained for the cases of integral molding of the microneedles and the base. However, various other cases may be provided.

Moreover, the first to seventh embodiments are explained for the cases of molding by arranging the three microneedles in a single row. However, the number of microneedles is not limited to three, and number of microneedles may be one, two, or four or more.

Moreover, the eighth to tenth embodiments are explained for the cases of arranging the microneedles in multiple rows, by using examples of square arrangement, circular arrangement, and radial arrangement. However, the present invention is not limited to these examples, and for example, a staggered arrangement, etc., may be provided.

Moreover, the direction of the slide pins is not limited to a single direction, and it is also possible to insert in multiple directions, and to adjust the orientations of resulting horizontal holes.

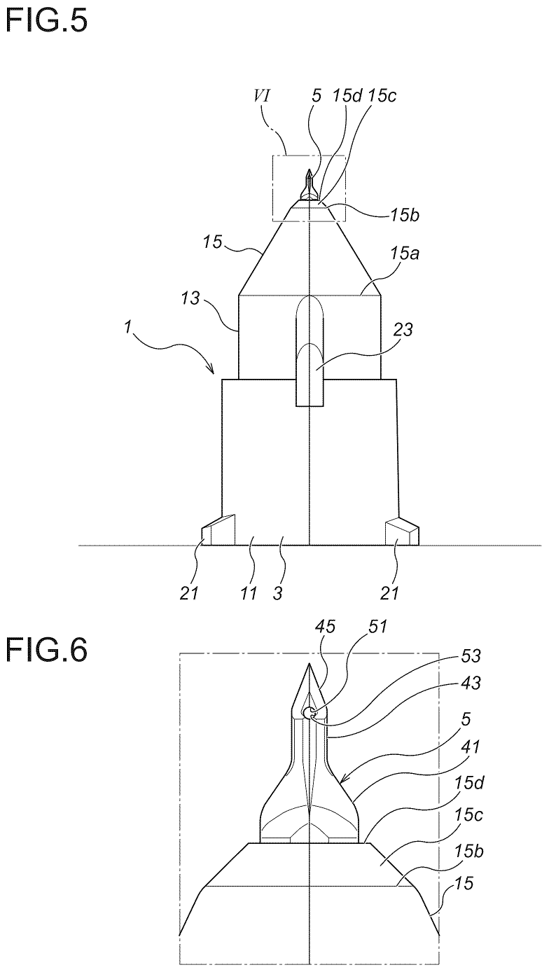

Moreover, in the first to seventh embodiments, with reference to FIG. 5, the microneedle is positioned at the center in the right and left directions. However, it is also possible to offset closer to any of right and left sides. Accordingly, inclined puncture can be performed easily.

Moreover, in a case of multiple molding of microneedles, it is possible to vary the length of each of microneedles non-uniformly.

Moreover, the first to tenth embodiments are explained, as the example, in the case that the cross section of the horizontal hole is in a circular shape. However, the present invention is not limited to that example, and it is also possible to be in a square shape, or in other shapes.

Moreover, with regard to the position of horizontal hole, it is also possible to provide in the neck section 43 with reference to FIG. 8.

Moreover, in the first embodiment, the horizontal hole is provided along the ridgeline of the pyramid shape. However, the present invention is not limited to this configuration, and it is also possible to provide the horizontal hole in a plane between the adjacent ridgelines.

Furthermore, the configurations in the drawings are shown for the illustrative purpose only.

### Industrial Applicability

The present invention relates to a microneedle manufacturing method, a microneedle, and a microneedle unit, and in particular, the present invention relates to those allowing easy manufacturing of a microneedle provided with a horizontal hole in a penetrated state serving as a medical solution discharge port. The present invention may be suitably applied, for example, to manufacturing of microneedles for vaccination.

### EXPLANATION OF REFERENCE NUMERALS AND SIGNS

1 Microneedle unit
1′ Microneedle unit
3 Needle base
3′ Base
5 Microneedle
41 Foundation
43 Neck section
45 Boring section
51 Horizontal hole
53 Medical solution discharge port
61 Vertical hole
73 Slide core
73a Cavity
73b Cavity
73c Recess
81 Core insert
83 Core pin
85 Slide pin

1. A microneedle manufacturing method, for manufacturing a microneedle provided with a vertical hole serving as a medical solution flow path, and a connected horizontal hole serving as a medical solution discharge port in a penetrated state, wherein:
a slide pin, for forming the horizontal hole in a mold, is caused to appear;
a core pin, for forming the vertical hole, is caused to abut on the appeared slide pin;
resin is filled in the mold in the appeared and abutting states; and
after passing of a curing period, the core pin is retracted, and also the slide pin is withdrawn, whereby:
the microneedle provided with the vertical hole serving as the medical solution flow path, and the connected horizontal hole serving as the medical solution discharge port in the penetrated state, is molded integrally.

2. The microneedle manufacturing method as claimed in claim 1, wherein:
the mold is configured by a pair of cores arranged to be joinable and separatable;
the slide pin is caused to appear in a clearance between the pair of cores in a state separate from each other;
the slide pin is sandwiched by moving the pair of cores close to each other;
the core pin is caused to appear and abut on the slide pin;
the resin is filled into the clearance between the pair of cores in the appeared and abutting states; and
after passing of the curing period, the core pin is retracted, and also the slide pin is withdrawn, whereby:
the microneedle provided with the vertical hole serving as the medical solution flow path, and the connected horizontal hole serving as the medical solution discharge port in the penetrated state, is molded integrally.

3. The microneedle manufacturing method as claimed in claim 2, wherein:
the pair of cores is a pair of slide cores arranged to be joinable and separatable in a direction orthogonal to an axis direction of the microneedle, and the microneedle is molded by this pair of slide cores.

4. The microneedle manufacturing method as claimed in claim 3, wherein:
a core insert is arranged between the pair of slide cores; and
the core pin appears and disappears by penetrating through the core insert.

5. The microneedle manufacturing method as claimed in claim 4, wherein:
in each of the pair of slide cores, a recess is formed for accommodating the slide pin, and a cavity is formed for accommodating the core insert in a state that the core pin is caused to appear.

6. The microneedle manufacturing method as claimed in claim 5, wherein:
the pair of slide cores is set to be in a size for molding an area extending from the microneedle to a needle base;
the core insert is also set to be in a size for molding the area extending from the microneedle to the needle base; and
the area extending from the microneedle to the needle base is molded integrally as a microneedle unit by using the pair of slide cores and the core insert.

7. The microneedle manufacturing method as claimed in claim 4, wherein:
a plurality of core pins is arranged in a single row in the core insert;
a cavity is provided in the slide core, for accommodating the core insert in a state that the plurality of core pins is caused to appear; and
a plurality of microneedles is molded integrally in a state that the plurality of microneedles is arranged in the single row by using the pair of slide cores and the core insert.

8. The microneedle manufacturing method as claimed in claim 7, wherein:
the horizontal holes of the plurality of microneedles are formed by single straight penetration of the single slide pin.

9. The microneedle manufacturing method as claimed in claim 2, wherein:
the pair of cores is a cavity insert upper plate and a cavity insert lower plate arranged to be joinable and separatable in an axis direction of the microneedle, and the microneedle is molded by the cavity insert upper plate and the cavity insert lower plate.

10. The microneedle manufacturing method as claimed in claim 9, wherein:
a core insert is arranged to be movable in the axis direction of the microneedle below the cavity insert lower plate.

11. A microneedle provided with a vertical hole serving as a medical solution flow path, and a connected horizontal hole serving as a medical solution discharge port in a penetrated state, and molded integrally.

12. The microneedle as claimed in claim 11, wherein:
a cross section of the horizontal hole is in a circular shape.

13. The microneedle as claimed in claim 11, wherein:
the microneedle includes a foundation, a neck section provided on a tip side of the foundation, and a boring section provided on a tip side of the neck section; and
the horizontal hole is provided in a lower part of the boring section.

14. The microneedle as claimed in claim 13, wherein:
the boring section is a pyramid section; and
the horizontal hole is provided by penetrating through a line connecting a pair of edges of the pyramid section.

15. The microneedle as claimed in claim 14, wherein:
an end part of each of the edges of the pyramid section on a side of the neck section is formed in an arc shape protruding outwardly.

16. The microneedle as claimed in claim 13, wherein:
a lower part of the foundation is formed to be in a larger diameter than that of the neck section, and the diameter decreases gradually toward the neck section.

17. The microneedle as claimed in claim 13, wherein:
the vertical hole is formed extending from the foundation to the neck section, and becomes narrower gradually from the foundation to the neck section.

18. A microneedle unit, provided with:
the microneedle as claimed in claim 11; and
a needle base, integrally molded on a base end side of the microneedle, and a tip of a cylindrical body of a syringe is coupled with the base end side.
